SEQUENCE LISTING (90 sequences)

SEQ ID NO: 1 - DNA, 27 bp, Artificial Sequence
  RNase H-dependent PCR primer
acactggctgcaacagcatcrcaggac

SEQ ID NO: 2 - DNA, 28 bp, Artificial Sequence
  RNase H-dependent PCR primer
ggataaacatctgtctctgcgrcattgg

SEQ ID NO: 3 - DNA, 27 bp, Artificial Sequence
  RNase H-dependent PCR primer
aacagaatggcctctctggcraatcgg

SEQ ID NO: 4 - DNA, 31 bp, Artificial Sequence
  RNase H-dependent PCR primer
gaacatcacagccacccagaccggragactg

SEQ ID NO: 5 - DNA, 27 bp, Artificial Sequence
  RNase H-dependent PCR primer
cttggagaaaggctcagttcraagtga

SEQ ID NO: 6 - DNA, 27 bp, Artificial Sequence
  RNase H-dependent PCR primer
ctgaggaaaccctctgtgcargtggac

SEQ ID NO: 7 - DNA, 27 bp, Artificial Sequence
  RNase H-dependent PCR primer
aagggaatcctctgactgtgraaatgg

SEQ ID NO: 8 - DNA, 31 bp, Artificial Sequence
  RNase H-dependent PCR primer
tccaccagttccttcaacttcaccratcact

SEQ ID NO: 9 - DNA, 27 bp, Artificial Sequence
  RNase H-dependent PCR primer
ttgataccaaagcccgtctcragcacg

SEQ ID NO: 10 - DNA, 31 bp, Artificial Sequence
  RNase H-dependent PCR primer
ttcatcaaaacccttggggacagcrtcatct

SEQ ID NO: 11 - DNA, 31 bp, Artificial Sequence
  RNase H-dependent PCR primer
gatggaaggtttacagcacagctcraatagt

SEQ ID NO: 12 - DNA, 31 bp, Artificial Sequence
  RNase H-dependent PCR primer
tcccagctcagcgattcagcctccrtacatg

SEQ ID NO: 13 - DNA, 32 bp, Artificial Sequence
  RNase H-dependent PCR primer
cacagtggaagattaagagtcacgcrttgact

SEQ ID NO: 14 - DNA, 30 bp, Artificial Sequence
  RNase H-dependent PCR primer
gcaaagctccctgtaccttacggrcctccg

SEQ ID NO: 15 - DNA, 31 bp, Artificial Sequence
  RNase H-dependent PCR primer
aatccgccaaccttgtcatctccgrcttcag

SEQ ID NO: 16 - DNA, 27 bp, Artificial Sequence
  RNase H-dependent PCR primer
accctgagtgtccaggagggrtgacat

SEQ ID NO: 17 - DNA, 27 bp, Artificial Sequence
  RNase H-dependent PCR primer
ctgaggaaaccctctgtgcarttggac

SEQ ID NO: 18 - DNA, 31 bp, Artificial Sequence
  RNase H-dependent PCR primer
cccagcaggcagatgattctcgttrattcgg

SEQ ID NO: 19 - DNA, 31 bp, Artificial Sequence; identical to SEQ ID NO: 18
  RNase H-dependent PCR primer

SEQ ID NO: 20 - DNA, 31 bp, Artificial Sequence
  RNase H-dependent PCR primer
tctggtatgtgcaataccccaaccraaggag

SEQ ID NO: 21 - DNA, 27 bp, Artificial Sequence
  RNase H-dependent PCR primer
ttacaaacgaagtggcctccrctgtta

SEQ ID NO: 22 - DNA, 27 bp, Artificial Sequence
  RNase H-dependent PCR primer
ggattgcgctgaaggaagagrctgcat

SEQ ID NO: 23 - DNA, 27 bp, Artificial Sequence
  RNase H-dependent PCR primer
aactgcacgtaccagacatcrtgggta

SEQ ID NO: 24 - DNA, 31 bp, Artificial Sequence
  RNase H-dependent PCR primer
tgaaggtcacctttgataccacccrttaaag

SEQ ID NO: 25 - DNA, 30 bp, Artificial Sequence
  RNase H-dependent PCR primer
cactgctgaccttaacaaaggcgragacaa

SEQ ID NO: 26 - DNA, 27 bp, Artificial Sequence
  RNase H-dependent PCR primer
aaggagaggacttcaccacgrtactgg

SEQ ID NO: 27 - DNA, 31 bp, Artificial Sequence
  RNase H-dependent PCR primer
tgcctcgctggataaatcatcaggracgtac

SEQ ID NO: 28 - DNA, 27 bp, Artificial Sequence
  RNase H-dependent PCR primer
aactgcacgtaccagacatcrtgggta

SEQ ID NO: 29 - DNA, 27 bp, Artificial Sequence
  RNase H-dependent PCR primer
gatagccatacgtccagatgrtgagtc

SEQ ID NO: 30 - DNA, 31 bp, Artificial Sequence
  RNase H-dependent PCR primer
tgccactcttaataccaaggagggrttacac

SEQ ID NO: 31 - DNA, 31 bp, Artificial Sequence; identical to SEQ ID NO: 20
  RNase H-dependent PCR primer

SEQ ID NO: 32 - DNA, 27 bp, Artificial Sequence
  RNase H-dependent PCR primer
gtcctgtcctcttgatagccrttataa

SEQ ID NO: 33 - DNA, 27 bp, Artificial Sequence
  RNase H-dependent PCR primer
agcaaaaacttcggaggcggraaataa

SEQ ID NO: 34 - DNA, 31 bp, Artificial Sequence
  RNase H-dependent PCR primer
accctgctgaaggtcctacattccrtgataa

SEQ ID NO: 35 - DNA, 27 bp, Artificial Sequence
  RNase H-dependent PCR primer
tcctggtgacagtagttacgrggtggt

SEQ ID NO: 36 - DNA, 27 bp, Artificial Sequence
  RNase H-dependent PCR primer
accctgagtgtccaggagggragacac

SEQ ID NO: 37 - DNA, 31 bp, Artificial Sequence
  RNase H-dependent PCR primer
aggctcaaagccttctcagcagggracgatt

SEQ ID NO: 38 - DNA, 31 bp, Artificial Sequence
  RNase H-dependent PCR primer
gatggaaggtttacagcacagctcraataat

SEQ ID NO: 39 - DNA, 31 bp, Artificial Sequence
  RNase H-dependent PCR primer
agcccagccatgcaggcatctaccrtctgtc

SEQ ID NO: 40 - DNA, 30 bp, Artificial Sequence
  RNase H-dependent PCR primer
ctccctgattctggagtccgccargcacct

SEQ ID NO: 41 - DNA, 31 bp, Artificial Sequence
  RNase H-dependent PCR primer
ccactctgaagatccagccctcagraaccct

SEQ ID NO: 42 - DNA, 33 bp, Artificial Sequence
  RNase H-dependent PCR primer
ctgtagccttgagatccaggctacgaragcttc

SEQ ID NO: 43 - DNA, 38 bp, Artificial Sequence
  RNase H-dependent PCR primer
ctaacattctcaactctgactgtgagcaacartgagcg

SEQ ID NO: 44 - DNA, 29 bp, Artificial Sequence
  RNase H-dependent PCR primer
tcgctcaggctggagtcggctgrctccca

SEQ ID NO: 45 - DNA, 38 bp, Artificial Sequence
  RNase H-dependent PCR primer
tcaaatttcactctgaagatccggtccacaaragctgc

SEQ ID NO: 46 - DNA, 35 bp, Artificial Sequence
  RNase H-dependent PCR primer
gataacttccaatccaggaggccgaacarcttcta

SEQ ID NO: 47 - DNA, 28 bp, Artificial Sequence
  RNase H-dependent PCR primer
ccctgaccctggagtctgccarggccca

SEQ ID NO: 48 - DNA, 27 bp, Artificial Sequence
  RNase H-dependent PCR primer
gctcaggctgctgtcggctgrctccca

SEQ ID NO: 49 - DNA, 33 bp, Artificial Sequence
  RNase H-dependent PCR primer
gctctgagctgaatgtgaacgccttgrttgctt

SEQ ID NO: 50 - DNA, 33 bp, Artificial Sequence
  RNase H-dependent PCR primer
gctctgagatgaatgtgagtgccttgrgagctc

SEQ ID NO: 51 - DNA, 31 bp, Artificial Sequence
  RNase H-dependent PCR primer
ccactctgacgattcagcgcacagragcagg

SEQ ID NO: 52 - DNA, 31 bp, Artificial Sequence
  RNase H-dependent PCR primer
ccactctcaagatccagcctgcagragcttc

SEQ ID NO: 53 - DNA, 29 bp, Artificial Sequence
  RNase H-dependent PCR primer
cccctcaagctggagtcagctgrctccca

SEQ ID NO: 54 - DNA, 31 bp, Artificial Sequence
  RNase H-dependent PCR primer
ctccctgtccctagagtctgccatrccccat

SEQ ID NO: 55 - DNA, 31 bp, Artificial Sequence
  RNase H-dependent PCR primer
gcatcctgaggatccagcaggtagrtgcgac

SEQ ID NO: 56 - DNA, 33 bp, Artificial Sequence
  RNase H-dependent PCR primer
gctctgagctgaatgtgaacgccttgrttgctc

SEQ ID NO: 57 - DNA, 28 bp, Artificial Sequence
  RNase H-dependent PCR primer
cactcaggctggtgtcggctgrctccca

SEQ ID NO: 58 - DNA, 38 bp, Artificial Sequence
  RNase H-dependent PCR primer
gctcacttaaatcttcacatcaattccctggragcttc

SEQ ID NO: 59 - DNA, 31 bp, Artificial Sequence; identical to SEQ ID NO: 41
  RNase H-dependent PCR primer

SEQ ID NO: 60 - DNA, 28 bp, Artificial Sequence
  RNase H-dependent PCR primer
ccctcacgttggcgtctgctgrtaccca

SEQ ID NO: 61 - DNA, 29 bp, Artificial Sequence
  RNase H-dependent PCR primer
cccctcactctggagtctgctgrcctcca

SEQ ID NO: 62 - DNA, 27 bp, Artificial Sequence
  RNase H-dependent PCR primer
gctggggttggagtcggctgrctccca

SEQ ID NO: 63 - DNA, 31 bp, Artificial Sequence
  RNase H-dependent PCR primer
ccactctgaagatccagcgcacagragcagc

SEQ ID NO: 64 - DNA, 31 bp, Artificial Sequence; identical to SEQ ID NO: 52
  RNase H-dependent PCR primer

SEQ ID NO: 65 - DNA, 33 bp, Artificial Sequence; identical to SEQ ID NO: 56
  RNase H-dependent PCR primer

SEQ ID NO: 66 - DNA, 30 bp, Artificial Sequence
  RNase H-dependent PCR primer
ccccctcactctggagtcagctarcccgca

SEQ ID NO: 67 - DNA, 33 bp, Artificial Sequence
  RNase H-dependent PCR primer
cttattccttcacctacacaccctgcragccac

SEQ ID NO: 68 - DNA, 28 bp, Artificial Sequence
  RNase H-dependent PCR primer
cgctcaggctggagttggctgrctccca

SEQ ID NO: 69 - DNA, 35 bp, Artificial Sequence
  RNase H-dependent PCR primer
cattctgaactgaacatgagctccttggragctgc

SEQ ID NO: 70 - DNA, 33 bp, Artificial Sequence
  RNase H-dependent PCR primer
gctctgagatgaatgtgagcaccttgrgagctc

SEQ ID NO: 71 - DNA, 30 bp, Artificial Sequence
  RNase H-dependent PCR primer
cactctgacgatccagcgcacacragcagc

SEQ ID NO: 72 - DNA, 28 bp, Artificial Sequence
  RNase H-dependent PCR primer
ccctgatcctggagtcgcccargcccct

SEQ ID NO: 73 - DNA, 33 bp, Artificial Sequence
  RNase H-dependent PCR primer
cttaaaccttcacctacacgccctgcragccac

SEQ ID NO: 74 - DNA, 33 bp, Artificial Sequence
  RNase H-dependent PCR primer
cttgtccactctgacagtgaccagtgrcccatg

SEQ ID NO: 75 - DNA, 30 bp, Artificial Sequence
  RNase H-dependent PCR primer
caccttggagatccagcgcacagragcagc

SEQ ID NO: 76 - DNA, 33 bp, Artificial Sequence
  RNase H-dependent PCR primer
gctctgagctgaatgtgaacgccttgrgagctc

SEQ ID NO: 77 - DNA, 30 bp, Artificial Sequence
  RNase H-dependent PCR primer
cagcctggcaatcctgtcctcagraaccgc

SEQ ID NO: 78 - DNA, 31 bp, Artificial Sequence; identical to SEQ ID NO: 41
  RNase H-dependent PCR primer

SEQ ID NO: 79 - DNA, 31 bp, Artificial Sequence
  RNase H-dependent PCR primer
ctactctgaaggtgcagcctgcagraactgc

SEQ ID NO: 80 - DNA, 29 bp, Artificial Sequence
  RNase H-dependent PCR primer
cctctcactgtgacatcggcccraaaagt

SEQ ID NO: 81 - DNA, 30 bp, Artificial Sequence
  RNase H-dependent PCR primer
cactctgacgatccagcgcacagragcagg

SEQ ID NO: 82 - DNA, 35 bp, Artificial Sequence
  RNase H-dependent PCR primer
gcactctgaactaaacctgagctctctgrgagctc

SEQ ID NO: 83 - DNA, 31 bp, Artificial Sequence
  RNase H-dependent PCR primer
ccactctgaagttccagcgcacacragcagc

SEQ ID NO: 84 - DNA, 30 bp, Artificial Sequence
  RNase H-dependent PCR primer
cctcctcactctggagtccgctarccagca

SEQ ID NO: 85 - DNA, 33 bp, Artificial Sequence
  RNase H-dependent PCR primer
ctctactctgaagatccagcgcacagragcagc

SEQ ID NO: 86 - DNA, 59 bp, Artificial Sequence
  RNase H-dependent PCR primer
taatgatacggcgaccaccgagatctacactctttccctacacgacgctcttccgatct

SEQ ID NO: 87 - DNA, 18 bp, Artificial Sequence
  Sequencing Adapter
gatcggaagagcacacgt

SEQ ID NO: 88 - DNA, 34 bp, Artificial Sequence
  Sequencing Adapter
gtgactggagttcagacgtgtgctcttccgatct

SEQ ID NO: 89 - DNA, 58 bp, Artificial Sequence
  Sequencing Adapter
aatgatacggcgaccaccgagatctacactctttccctacacgacgctcttccgatct

SEQ ID NO: 90 - DNA, 60 bp, Artificial Sequence
  Sequencing Adapter
caagcagaagacggcatacgagatcgtccattgtgactggagttcagacgtgtgctcttc

(19) United States
(12) Patent Application Publication
(10) Pub. No.: US 2022/0112539 A1
(43) Pub. Date: Apr. 14, 2022

(54) Methods For Targeted Complementary DNA Enrichment

(71) Applicants: National University of Singapore, Singapore (SG); Agency for Science, Technology and Research, Singapore (SG)

(72) Inventors: Jonathan Scolnick, Singapore (SG); Wang Yingting, Singapore (SG); Shawn Hoon, Singapore (SG)

(21) Appl. No.: 17/417,680

(22) PCT Filed: Dec. 26, 2019
(86) PCT No.: PCT/IB2019/001398

§ 371 Date: Jun. 23, 2021

Related U.S. Application Data
(60) Provisional application No. 62/785,916, filed on Dec. 28, 2018.

(51) Int. Cl. C12Q 1/6806; C12Q 1/6853

(52) U.S. Cl. CPC C12Q 1/6806; C12Q 1/6853

(57) ABSTRACT

The present invention provides methods for enriching a target complementary DNA (cDNA), comprising: (a) providing a plurality of cDNAs, each comprising a first universal sequence at an end, and wherein the plurality of cDNAs comprises the target cDNA; (b) amplifying the target cDNA with a universal forward primer complementary to the first universal sequence and a gene specific reverse primer, and wherein a second universal sequence is added to an end of the cDNA opposite the first universal sequence, by a nucleic acid amplification reaction, by ligation, or by a primer extension reaction; and (c) amplifying the amplicons or extension products using the universal forward primer and a universal reverse primer complementary to the second universal sequence. In one embodiment, the universal forward primer, the gene specific reverse primer and the second universal reverse primer are provided in the same reaction mixture such that the amplifying is a single step.

### RELATED APPLICATION

This application claims the benefit of U.S. Provisional Application No. 62/785,916, filed on Dec. 28, 2018. The entire teachings of the above application are incorporated herein by reference.

### BACKGROUND

The ability to enrich samples for specific nucleotides or proteins is important for both molecular biology research and medical applications. In particular, there are specific needs in single cell RNA sequencing for obtaining sequence information or tag counts from specific genes.

### SUMMARY OF THE INVENTION

The present invention provides methods for enriching a target complementary DNA (cDNA).

In various aspects, the invention provides methods of enriching a target cDNA, comprising the steps of:
(a) providing a plurality of cDNAs, wherein each cDNA comprises a first universal sequence at an end and wherein the plurality of cDNAs comprises the target cDNA to be enriched;
(b) providing a first reaction mixture comprising:
   at least one gene specific primer (e.g., at least one gene specific reverse primer) comprising a sequence that is complementary to all or a portion of a sequence in the target cDNA, and further comprises at least one second universal sequence at an end of the at least one gene specific primer;
(c) contacting the plurality of cDNAs with the first reaction mixture such that the at least one gene specific primer hybridizes with the target cDNA;
(d) extending the at least one gene specific primer to obtain at least one extension product;
(e) providing a second reaction mixture comprising:
   1) a universal oligonucleotide forward primer comprising a sequence that is complementary to all or a portion of the first universal sequence; and
   2) a universal oligonucleotide reverse primer comprising a sequence that is complementary to all or a portion of the at least one second universal sequence;
(f) contacting the at least one extension product with the second reaction mixture; and
(g) amplifying the at least one extension product, thereby enriching for the target cDNA.

In further aspects, the invention provides methods of enriching a target complementary DNA (cDNA). The methods generally comprise the steps of:
(a) providing a plurality of different cDNAs, wherein each cDNA comprises a first universal sequence at an end, and wherein the plurality of cDNAs comprises the target cDNA to be enriched;
(b) providing a first reaction mixture comprising:
   1) a universal oligonucleotide forward primer comprising a sequence that is complementary to all or a portion of the first universal sequence, and
   2) at least one gene specific reverse primer, wherein the at least one gene specific reverse primer comprises a sequence that is complementary to all or a portion of a sequence in the target cDNA, and further comprises at least one second universal sequence at an end of the at least one gene specific primer;
(c) contacting the plurality of cDNAs with the first reaction mixture;
(d) amplifying the target cDNA to obtain an amplicon;
(e) providing a second reaction mixture comprising:
   1) a universal oligonucleotide forward primer comprising a sequence that is complementary to all or a portion of the first universal sequence, and
   2) a universal oligonucleotide reverse primer, wherein the universal oligonucleotide reverse primer comprises a sequence that is complementary to all or a portion of the at least one second universal sequence; thereby enriching for the target cDNA;
(f) contacting the amplicon with the second reaction mixture; and
(g) amplifying the amplicon, thereby enriching for the target cDNA.

In additional aspects, the methods of enriching a target cDNA generally comprise the steps of:
(a) providing a plurality of different cDNAs, wherein each cDNA comprises a first universal sequence at an end, and wherein the plurality of cDNAs comprises the target cDNA to be enriched;
(b) providing a reaction mixture comprising:
   1) a universal oligonucleotide forward primer comprising a sequence that is complementary to all or a portion of the first universal sequence,
   2) at least one gene specific reverse primer, wherein the at least one gene specific reverse primer comprises a sequence that is complementary to all or a portion of a sequence in the target cDNA, and further comprises at least one second universal sequence at an end of the at least one gene specific primer, and
   3) a universal oligonucleotide reverse primer, wherein the universal oligonucleotide reverse primer comprises a sequence that is complementary to all or a portion of the at least one second universal sequence;
(c) contacting the plurality of cDNAs with the reaction mixture; and
(d) amplifying the target cDNA, thereby enriching for the target cDNA.

In other aspects, the invention provides methods of enriching a target cDNA, comprising the steps of:
(a) providing a plurality of cDNAs, wherein each cDNA comprises a first universal sequence at an end, and wherein the plurality of cDNAs comprises the target cDNA to be enriched;
(b) providing a first reaction mixture comprising:
   1) a first universal oligonucleotide forward primer comprising a sequence that is complementary to all or a portion of the first universal sequence, and
   2) a gene specific reverse primer comprising a sequence that is complementary to a sequence in the target cDNA;
(c) contacting the plurality of cDNAs with the first reaction mixture;
(d) amplifying the target cDNA to obtain a first amplicon;
(e) adding at least one second universal sequence to an end of each target cDNA in the first amplicon to obtain a conjugated amplicon;
(f) providing a second reaction mixture comprising:
   1) a second universal oligonucleotide forward primer comprising a sequence that is complementary to all or a portion of the first universal sequence, and
   2) a universal oligonucleotide reverse primer comprising a sequence that is complementary to all or a portion of the at least one second universal sequence;
(g) contacting the conjugated amplicon with the second reaction mixture; and
(h) amplifying the conjugated amplicon, thereby enriching for the target cDNA.

In particular embodiments of the various aspects of the invention, each cDNA in the plurality of cDNAs further comprises a cell identification tag or a unique molecular identifier (UMI) sequence, or a combination thereof. In some embodiments, the plurality of cDNAs is obtained by reverse transcribing mRNA from a single cell.

### BRIEF DESCRIPTION OF THE DRAWINGS

The present invention will now be described in detail with reference to the following figures which show:

FIG. 1 illustrates the general steps in an example method for producing a 3′ tagged cDNA library. In this example, the 3′ tag includes a universal sequence, a cell identification tag (Cell ID), and a unique molecular identifier (UMI) sequence. The same process can be utilized for creating 5′ tagged cDNA libraries. The oligos can be attached to beads, as shown.

FIG. 2 illustrates an example method of the invention for enriching a target cDNA utilizing a tagged cDNA library and a gene specific primer having a universal sequence at an end of the primer. Subsequently another PCR may be performed with the first universal sequence and second universal sequence (e.g., library amplification using two universal primers) in order to prepare for sequencing.

FIG. 3 illustrates an example method of the invention for enriching a target cDNA utilizing a tagged cDNA library, a gene specific primer lacking a universal sequence, and a ligated universal sequence. Subsequently, the amplicon can be ligated to a sequencing specific sequence (e.g., an Illumina® P7 sequence). Following addition of the sequencing specific sequence, the DNA may be PCR amplified using universal primers to enrich for ligated or tagmented DNAs.

FIG. 4 illustrates an example method of the invention where one end of the universal oligonucleotide forward primer has a blocking group, thereby blocking phosphorylation of the universal oligonucleotide forward primer on the one end. The T/A ligation is optional, but can improve ligation efficiency. For the ligation, only one strand is required for ligation, and in this scenario, only the bottom strand ligates, because the top strand of the ligating adaptor does not have a 5′ phosphate for ligation.

FIG. 5 illustrates an example sequencing method of the invention that may be utilized following the method of enrichment illustrated in FIG. 3 or FIG. 4.

FIG. 6 illustrates an example method of the invention utilizing a 3′ tagged cDNA library. Only one strand of cDNA is shown; however, the cDNA can be PCR amplified by two universal primers prior to using a gene-specific primer (GSP) for targeting (not shown). Therefore, it is double stranded at the point where the gene specific assay is performed (not shown). Alternatively, the same assay could be run prior to amplifying with the two universal primers.

FIG. 7 illustrates an example method of the invention to target multiple regions along one cDNA.

FIG. 8 shows a PCR result on a 1.2% agarose EtBr gel utilizing TCR alpha (TCRA) (Lane 2) and TCR beta (TCRB) (Lane 3) primers. Lane 1 shows the 1 kb MW markers.

FIG. 9 shows a PCR result for amplification of TCR alpha (TCRA) and TCR beta (TCRB) RNAs on an agarose EtBr gel. Lane 1 shows the 1 kb MW markers; Lane 2 shows TCR alpha ligation reaction; Lane 3 shows TCR beta ligation reaction; Lane 4 shows the TCR alpha PCR product; Lane 5 shows the TCR beta PCR product; and Lane 6 shows a control PCR product using no template.

FIG. 10 is a stained agarose gel showing PCR products obtained using individual (non-pooled) TCR alpha and TCR beta primers.

FIG. 11 is a stained agarose gel showing PCR products obtained using pooled TCR alpha (TCRa) primers and TCR beta (TCRb) primers, respectively. The right column contains a 1 kb plus ladder (ThermoFisher Scientific, Waltham, Mass.).

### DETAILED DESCRIPTION OF THE INVENTION

A description of example embodiments follows.

Unless defined otherwise, all technical and scientific terms used herein have the same meaning as commonly understood by one of ordinary skill in the art to which this invention pertains.

It should be noted that throughout this specification the term “comprising” is used to denote that embodiments of the invention “comprise” the noted features and as such, may also include other features. However, in the context of this invention, the term “comprising” may also encompass embodiments in which the invention “consists essentially of” the relevant features or “consists of” the relevant features.

The term “nucleotide” refers to naturally occurring ribonucleotide or deoxyribonucleotide monomers, as well as non-naturally occurring derivatives and analogs thereof. Accordingly, nucleotides can include, for example, nucleotides comprising naturally occurring bases (e.g., A, G, C, or T) and nucleotides comprising modified bases (e.g., 7-deazaguanosine, or inosine).

The term “sequence” in reference to a nucleic acid, refers to a contiguous series of nucleotides that are joined by covalent bonds (e.g., phosphodiester bonds).

The present invention provides methods for enriching one or more target complementary DNA (cDNA) within a pool of cDNAs from, e.g., a sequencing library.

As used herein, “complementary DNA” or “cDNA” refers to a nucleic acid molecule synthesized from a single-stranded RNA (e.g., messenger RNA (mRNA) or microRNA) template in a reaction catalyzed by a reverse transcriptase enzyme.

The methods described herein can apply to multiple cells (a plurality of cells) or to a single cell. In one embodiment, the methods apply to specific cDNAs within a pool of cDNAs from a single cell sequencing library. In some embodiments, the cDNAs or “plurality of cDNAs” are obtained by reverse transcribing mRNA from a single cell. In other embodiments, the cDNAs are obtained by reverse transcribing mRNA from multiple cells (pooled cells). In some embodiments, reverse transcription of the one or more target cDNA is part of a cDNA library generation process. The cDNA library can be a general cDNA library (e.g., a cDNA library for total mRNA from a cell) or a targeted cDNA library.

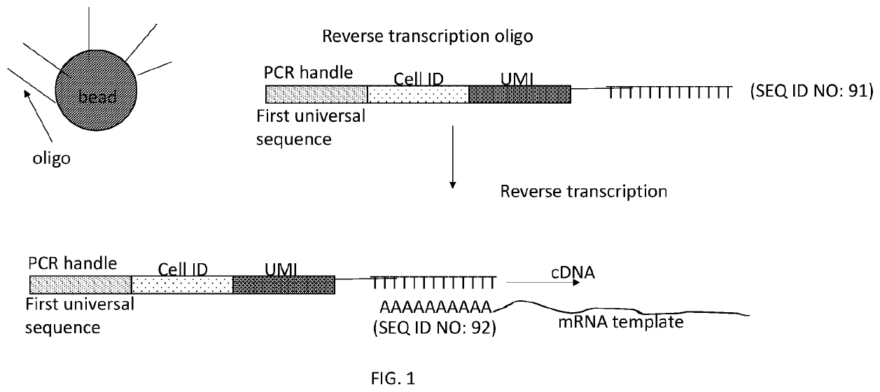

An example of a method of generating a cDNA library useful in the methods of the present invention is illustrated in FIG. 1. The method illustrated in FIG. 1 shows the generation of a 3′ tagged cDNA library, wherein the 3′ tag includes a first universal sequence (or a PCR handle), a cell identification tag (or a cell ID), and a UMI (or unique molecular identifier). The methods of the present invention can also be applied to a 5′ tagged cDNA library having a universal sequence (or a PCR handle), a cell identification tag (or a cell ID), and a UMI (or unique molecular identifier) on the 5′ end of the cDNAs of the library. As shown, the reverse transcription oligo may be bound to a bead and contacted with mRNA from a cell or pool of cells and the necessary polymerases to cause reverse transcription. The resultant cDNAs contain the first universal sequence, cell ID and UMI provided by the reverse transcription oligo.

As used herein, a “cell identification tag” refers to a sequence of nucleotides that can be incorporated into extension products (e.g., amplicons) and used in sequencing applications to identify the particular cell (e.g., a single cell) or cell type in which the extension product(s) was generated. A cell identification tag can be included in a primer (e.g., an extension primer, such as an oligo(dT) primer, or an amplification primer) for introduction into an extension product (e.g., a RT product, an amplicon). A cell identification tag can be incorporated into an extension product by a suitable nucleic acid polymerase, such as a reverse transcriptase enzyme or a DNA polymerase enzyme.

“Unique molecular identifiers” or “UMIs”, which are also called “Random Molecular Tags (RMTs),” are sequences of nucleotides that are used to tag a nucleic acid molecule (e.g., prior to amplification) and aid in the identification of duplicates. UMIs are generally random sequences and typically range in size from about 4 to about 20 nucleotides in length. Examples of UMIs are known in the art.

In some embodiments, both target and non-target nucleic acid molecules (i.e., mRNAs) in a sample (e.g., single cell) are reverse transcribed, thereby producing target cDNA products and non-target cDNA products.

The term “cells” encompasses mammalian cells, plant cells, bacterial cells and fungal cells.

The methods described herein can be performed using standard laboratory equipment, e.g., a well (e.g., microwell or nanowell) on a plate or in an array. The well can further contain oligonucleotides (e.g., primers), which can be immobilized on beads.

The methods described herein utilize nucleic acid amplification techniques, such as polymerase chain reaction (PCR). In some embodiments, methods described utilize RNase H-dependent PCR (rhPCR). In rhPCR, the 3′ end of a PCR oligonucleotide primer contains a blocking domain that is separated from the primer by a single RNA base. When the primer hybridizes to its target, RNAse H is able to cleave the RNA base, releasing the blocking domain and allowing polymerization starting from the primer. This activity reduces primer dimer formation, which is important in large multiplexing PCR reactions, such as in, e.g., a T-cell receptor (TCR) reaction which requires ˜30 primers each to amplify all of the possible alpha and beta chains.

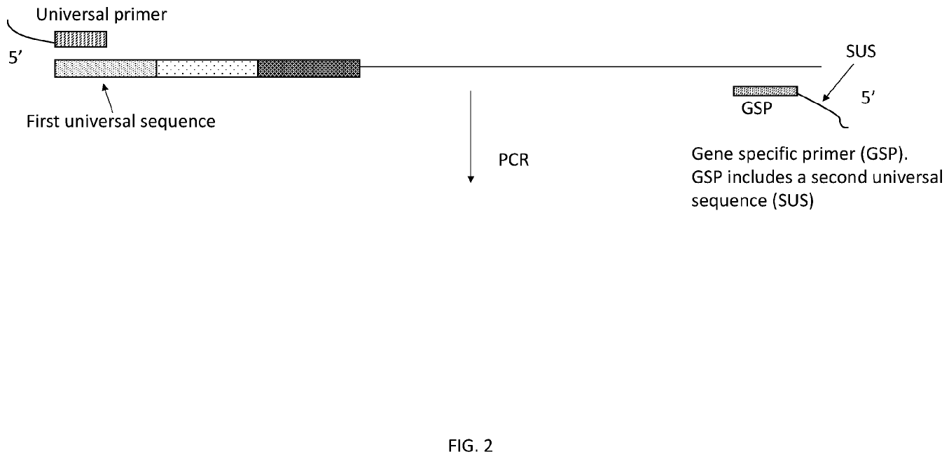

In a first aspect, the invention provides a method of enriching a target cDNA, as illustrated in FIG. 2. The method generally comprises the steps of:
(a) providing a plurality of different cDNAs, wherein each cDNA comprises a first universal sequence at an end, and wherein the plurality of cDNAs comprises the target cDNA to be enriched;
(b) providing a first reaction mixture comprising:
   1) a first universal oligonucleotide forward primer comprising a sequence that is complementary to all or a portion of the first universal sequence, and
   2) at least one gene specific reverse primer, wherein the at least one gene specific reverse primer comprises a sequence that is complementary to all or a portion of a sequence in the target cDNA, and further comprises at least one second universal sequence at an end of the at least one gene specific primer;
(c) contacting the plurality of cDNAs with the first reaction mixture;
(d) amplifying the target cDNA to obtain an amplicon;
(e) providing a second reaction mixture comprising:
   1) a second universal oligonucleotide forward primer comprising a sequence that is complementary to all or a portion of the first universal sequence, and
   2) a universal oligonucleotide reverse primer, wherein the universal oligonucleotide reverse primer comprises a sequence that is complementary to all or a portion of the at least one second universal sequence; thereby enriching for the target cDNA;
(f) contacting the amplicon with the second reaction mixture; and
(g) amplifying the amplicon, thereby enriching for the target cDNA.

“Target complementary DNA” or “target cDNA,” as used herein, refers to a specific cDNA within a pool of cDNAs that is being enriched by the methods described. The target cDNA may be expressed at low levels, and it may be beneficial to artificially increase the copy number (i.e., enrich) for analyzing (e.g., sequencing) the target cDNA within the larger pool.

In the first aspect of the invention described above, the first universal primer and the second universal primer, in some embodiments, can be the same primer or, in other embodiments, can be different primers, provided that each of the different primers comprises a sequence that is complementary to all or a portion of the first universal sequence.

This first aspect of the invention allows for the scenario where, prior to or following a total cDNA amplification step, a portion of the cDNA can be removed and specifically used as input for a targeted PCR reaction to enrich for the target cDNA.

In a second aspect, the invention provides a method of enriching a target cDNA comprising the steps of:
(a) providing a plurality of different cDNAs, wherein each cDNA comprises a first universal sequence at an end, and wherein the plurality of cDNAs comprises the target cDNA to be enriched;
(b) providing a reaction mixture comprising:
   1) a universal oligonucleotide forward primer comprising a sequence that is complementary to all or a portion of the first universal sequence,
   2) at least one gene specific reverse primer, wherein the at least one gene specific reverse primer comprises a sequence that is complementary to all or a portion of a sequence in the target cDNA, and further comprises at least one second universal sequence at an end of the at least one gene specific primer, and
   3) a universal oligonucleotide reverse primer, wherein the universal oligonucleotide reverse primer comprises a sequence that is complementary to all or a portion of the at least one second universal sequence;
(c) contacting the plurality of cDNAs with the reaction mixture; and
(d) amplifying the target cDNA, thereby enriching for the target cDNA.

In the second aspect, the gene specific reverse primer and the universal oligonucleotide reverse primer are provided in the same reaction mixture such that the amplifying is a single step. In one scenario, the gene specific primer is added to a total cDNA amplification PCR step so that all cDNAs are amplified and some are specifically amplified to ensure that they do not drop out of the assay.

In some embodiments, each cDNA in the plurality of cDNAs further comprises a cell identification tag or a unique molecular identifier (UMI) sequence, or a combination thereof. The cell identification tag or UMI sequence, or combination thereof, can further comprise a poly(T) sequence. In some embodiments, the cell identification tag or the UMI sequence, or the combination thereof, are preserved after the target cDNA is amplified.

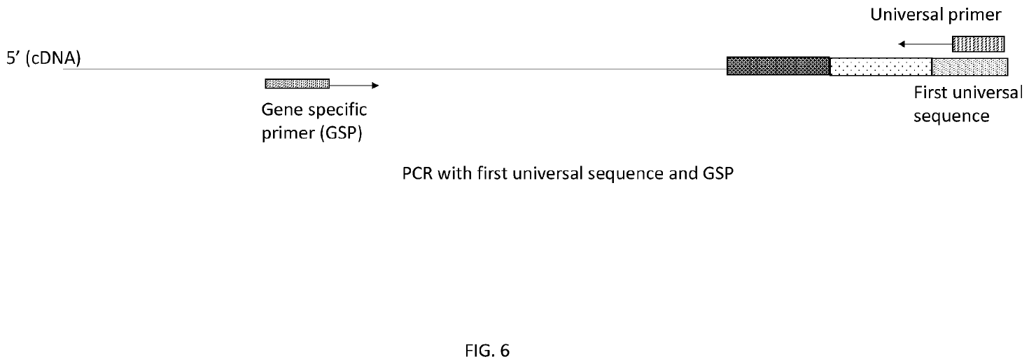

In various embodiments, the first universal sequence is at the 5′ end of each cDNA molecule, and in other embodiments, the first universal sequence is at the 3′ end of each cDNA molecule (FIG. 6). For example, the same method can be performed with the initial strand flipped, e.g. using the 10× genomics 5′ VdJ assay, where the cell identification tag is added to the growing 3′ end of the cDNA rather than the 5′ end of the cDNA.

In some embodiments, the amplifying of the target cDNA in step (d) of the aforementioned aspect of the invention comprises amplifying by polymerase chain reaction (PCR). In other embodiments, the amplifying of the target cDNA in step (d) of the aforementioned aspect of the invention comprises amplifying by a non-PCR based amplification method such as, for example, by loop mediated isothermal amplification (LAMP).

In some embodiments, the at least one gene specific reverse primer further comprises a blocking domain separated from the primer by an RNA base. When the at least one gene specific reverse primer comprises the blocking domain separated from the primer by an RNA base, the amplifying of the target cDNA in step (d) can be by rhPCR.

In some embodiments, the methods further comprise sequencing one or more portions of the target cDNA using the universal oligonucleotide forward primer, the universal oligonucleotide reverse primer, the at least one gene specific reverse primer, or a combination thereof.

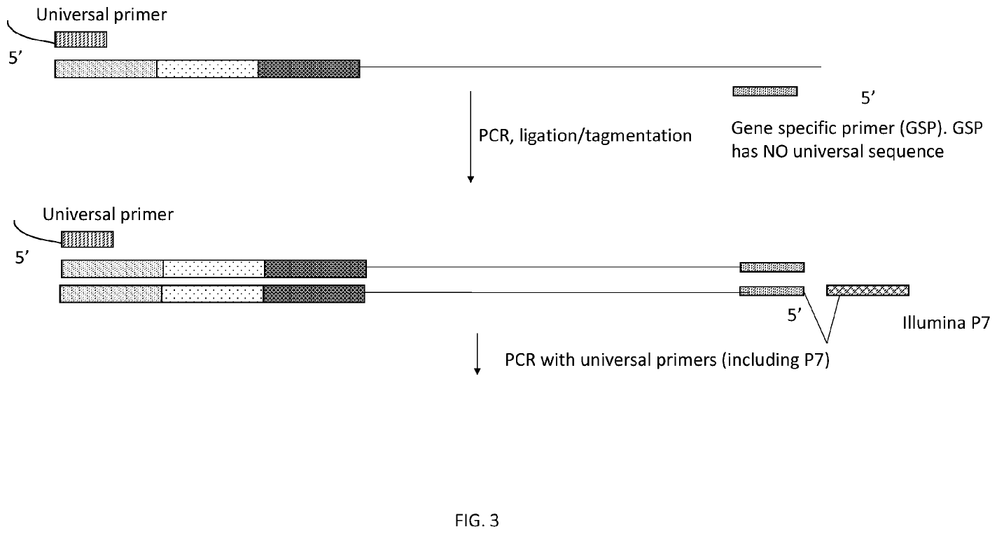

In another aspect, the invention provides a method of enriching a target cDNA, an example of which is illustrated in FIG. 3, comprising the steps of:
(a) providing a plurality of cDNAs, wherein each cDNA comprises a first universal sequence at an end, and wherein the plurality of cDNAs comprises the target cDNA to be enriched;
(b) providing a first reaction mixture comprising:
   1) a universal oligonucleotide forward primer comprising a sequence that is complementary to all or a portion of the first universal sequence, and
   2) a gene specific reverse primer comprising a sequence that is complementary to a sequence in the target cDNA;
(c) contacting the plurality of cDNAs with the first reaction mixture;
(d) amplifying the target cDNA to obtain a first amplicon;
(e) adding at least one second universal sequence to an end of each target cDNA in the first amplicon to obtain a conjugated amplicon;
(f) providing a second reaction mixture comprising:
   1) a universal oligonucleotide forward primer comprising a sequence that is complementary to all or a portion of the first universal sequence, and
   2) a universal oligonucleotide reverse primer comprising a sequence that is complementary to all or a portion of the at least one second universal sequence;
(g) contacting the conjugated amplicon with the second reaction mixture; and
(h) amplifying the conjugated amplicon, thereby enriching for the target cDNA.

In some embodiments, each cDNA in the plurality of cDNAs further comprises a cell identification tag or a UMI sequence, or a combination thereof. The cell identification tag or UMI sequence, or combination thereof, can further comprise a poly(T) sequence. In some embodiments, the cell identification tag or the UMI sequence, or the combination thereof, are preserved after the conjugated amplicon of target cDNA is amplified.

In various embodiments, the first universal sequence is at the 5′ end of each cDNA molecule, and in other embodiments, the first universal sequence is at the 3′ end of each cDNA molecule.

In some embodiments, at least one of the amplifying steps of the aforementioned aspect of the invention comprises amplifying by polymerase chain reaction.

In some embodiments, the gene specific reverse primer further comprises a blocking domain separated from the primer by an RNA base. When the gene specific reverse primer comprises the blocking domain separated from the primer by an RNA base, at least one of the amplifying steps can be by an RNase H-dependent polymerase chain reaction.

In some embodiments, the at least one second universal sequence (e.g., an Illumina® P7 sequence) is ligated to an end of the first amplicon opposite the first universal sequence (e.g., an Illumina® p5 sequence) as illustrated in FIG. 3. In some embodiments, the at least one second universal sequence is added using transposons in accordance with standard techniques known to those of skill in the art (e.g., tagmentation). In other embodiments, the at least one second universal sequence is added by fragmenting nucleic acid molecules in the first amplicon and ligating the at least one second universal sequence to the fragments, by a primer extension reaction, or by a nucleic acid amplification reaction, or a combination thereof. Ligation can be limited to the gene specific primer end only, because the universal primer side already contains proper sequencing specific sequences (e.g., Illumina® P5 sequence). The amplicon may also be shortened by fragmentation followed by ligation as would be understood in the art, or the amplicon may be used as a substrate for tagmentation or other methods for adding a sequencing specific sequence. In some embodiments, the at least one second universal sequence is added by ligating, by a primer extension reaction, or by a nucleic acid amplification reaction, or a combination thereof, the at least one second universal sequence to an end of each target cDNA in the first amplicon at an end opposite to the at least one first universal sequence.

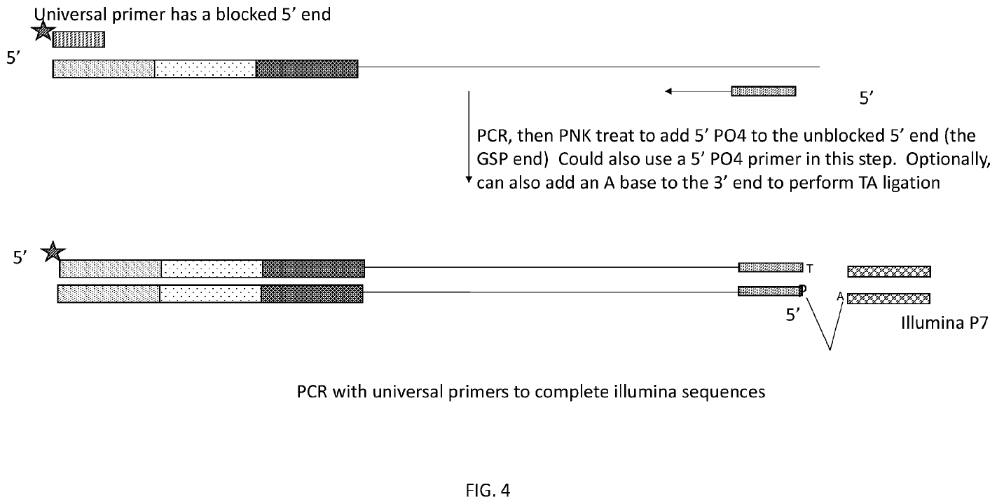

In some embodiments, one end of the universal oligonucleotide forward primer has a blocking group, thereby blocking phosphorylation of the universal oligonucleotide forward primer on the one end, as illustrated in FIG. 4. In some embodiments, the blocking group is an inverted dTTP and/or at the 5′ end of the universal oligonucleotide forward primer.

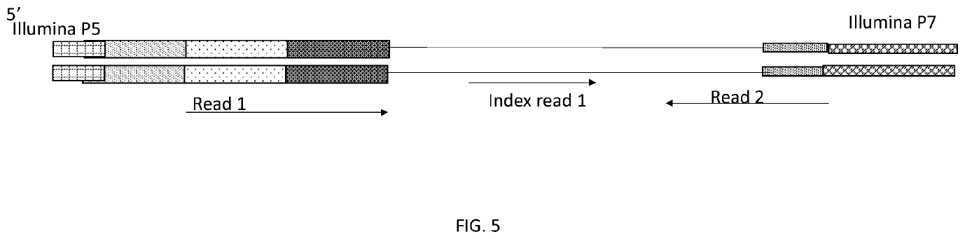

In further embodiments, the methods further comprise sequencing one or more portion of the target cDNA using the universal oligonucleotide forward primer, the universal oligonucleotide reverse primer, the gene specific reverse primer, a gene specific forward primer, or a combination thereof. An exemplary illustration of a sequencing technique useful in the methods herein is shown in FIG. 5.

Sequencing can be normal Illumina® sequencing or can use gene specific primers (or a mix of both). In the case where a long region of the amplicon is needed and the amplicon is not digested, a gene specific primer can be used in place of any of the standard sequencing primers. In the example illustrated in FIG. 5, a normal read 1 is performed to collect the cell identifier and UMI sequences. There may be a reason to not sequence part of the DNA, such as in TCR sequences, the continuation of read 1 would lead to common sequence that is not useful for informational purposes. Therefore, the index read can use a gene specific primer and sequence additional bases that have useful information content. Subsequently, the Illumina® read 2 can be done with standard primers (or another gene specific primer) to sequence additional useful sequence. For example, with TCR, useful sequence data must cover all of the recombined segments. In this example, read 1 would just read into the constant region, so instead a gene specific primer is utilized that hybridizes to the constant region in order to skip the intervening sequence and identify the J region sequence. Read 2 then starts with the V region and sequences through the D region.

Other items to note, especially around TCR: (1) The index read sequence in this illustration will start with a number of bases that are common to all of the amplicons. They must either be sequenced along with other cDNAs to balance the base composition (for Illumina® sequencing specifically) or a synthetic base balancing set of DNAs must be included in the sequencing run that are matched specifically to balance the bases from the common TCR region. (2) It may be useful for the TCR (in this case) to have a mutated or missing standard Illumina® index read primer binding site so that the standard Illumina® index read primer does not hybridize to these specific amplicons. This way, the gene specific primer and index read primer can be mixed together and the TCR can be sequenced along with other cDNAs.

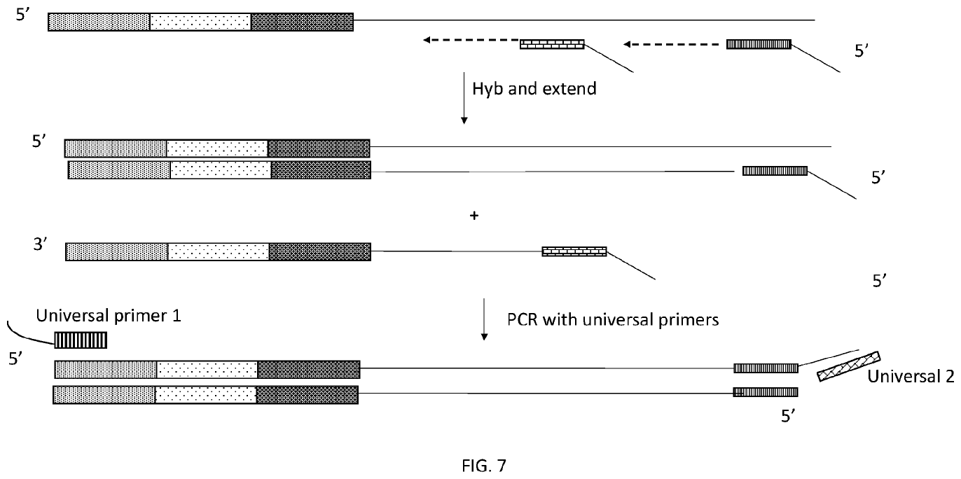

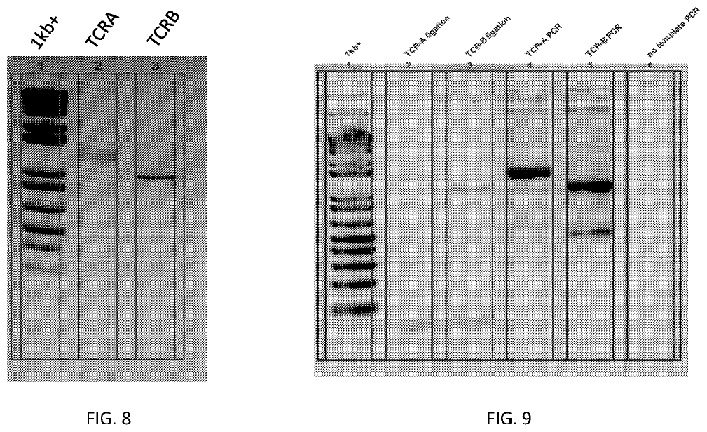

In other aspects, the invention provides methods of enriching a target cDNA, an example of which is illustrated in FIG. 7, comprising the steps of:
(a) providing a plurality of cDNAs, wherein each cDNA comprises a first universal sequence at an end and wherein the plurality of cDNAs comprises the target cDNA to be enriched;
(b) providing a first reaction mixture comprising:
   at least one gene specific reverse primer comprising a sequence that is complementary to all or a portion of a sequence in the target cDNA, and further comprises at least one second universal sequence at an end of the at least one gene specific primer;
(c) contacting the plurality of cDNAs with the first reaction mixture such that the at least one gene specific reverse primer hybridizes with the target cDNA;
(d) extending the at least one gene specific reverse primer to obtain at least one extension product;
(e) providing a second reaction mixture comprising:
   1) a universal oligonucleotide forward primer comprising a sequence that is complementary to all or a portion of the first universal sequence; and
   2) a universal oligonucleotide reverse primer comprising a sequence that is complementary to all or a portion of the at least one second universal sequence;
(f) contacting the at least one extension product with the second reaction mixture; and
(g) amplifying the at least one extension product, thereby enriching for the target cDNA.

In some embodiments, each cDNA in the plurality of cDNAs further comprises a cell identification tag or a UMI sequence, or a combination thereof. The cell identification tag or UMI sequence, or combination thereof, can further comprise a poly(T) sequence. In some embodiments, the cell identification tag or the UMI sequence, or the combination thereof, are preserved after the at least one extension product is amplified.

In various embodiments, the first universal sequence is at the 5′ end of each cDNA molecule, and in other embodiments, the first universal sequence is at the 3′ end of each cDNA molecule.

In some embodiments, the extension step (d) utilizes a polymerase that is deficient in flap endonuclease activity. In other embodiments, a standard thermostable DNA polymerase, which includes flap endonuclease activity, is utilized in extension step (d).

In some embodiments, the step of amplifying the at least one extension product in (g) comprises amplifying by polymerase chain reaction.

As shown in FIG. 7, multiple probes for multiple genes and possibly multiple probes hybridizing along the same cDNA (shown) can be hybridized to cDNA and extended. Then, the reaction is cleaned up and PCR amplified using common primers. This has an advantage of being able to target multiple regions along one cDNA. An alternative embodiment can include hybridizing one probe per cDNA, amplifying and fragmenting for Illumina® sequencing as described previously herein. Another embodiment can involve hybridizing one probe per gene and using it as a template for long read sequencing, such as Oxford nanopore technology.

In further embodiments, the methods further comprise sequencing one or more portion of the target cDNA using the universal oligonucleotide forward primer, the universal oligonucleotide reverse primer, the gene specific reverse primer, a gene specific forward primer, or a combination thereof. In other embodiments, the methods further comprise the step of sequencing the enriched target cDNA.

In some embodiments of the aspects described herein, the gene specific primer may contain modified bases to increase specificity. For example, rh-PCR can be used with the gene specific primer. Pools of gene specific primers can be used rather than just one, for example, primer pools that can bind all (or most) TCR V regions.

In some embodiments of the aspects described herein, ligation methods may include using a 5′ blocked universal primer in the first PCR (e.g., a 5′ inverted dT) and then using a 5′ phosphorylated gene specific primer. This can then be ligated only on one end by a ligation adaptor that is not phosphorylated. Alternatively, instead of using a phosphorylated gene specific primer, a 5′ phosphate can be added to the primer before or after PCR using PNK. Furthermore, digestion can take place by sheering (e.g., covaris) or enzymatically (e.g., exo or endo nuclease treatment). DNA can be digested to different lengths so that sequencing is able to sequence across all of the necessary regions of interest of a target cDNA and then assembling those different length reads bioinformatically.

The following features and related benefits can be obtained using the methods described herein:
(1) Enrichment of particular cDNAs within a pool of cDNAs from single cell sequencing libraries—Particular RNAs are expressed at low levels, and it is beneficial to artificially increase the copy number for sequencing those particular cDNAs within the larger pool, while still sequencing the larger pool;
(2) Single targeting primer with universal primer for amplification—Targets cDNA of interest at the 5′ end while maintaining any cell identifying sequences added to the 3′ end of the cDNA;
(3) T cell receptor sequencing from a 3′ tagged cDNA—When making single cell RNA-sequencing libraries, it may be beneficial to use 3′ tag sequencing; however, then information such as the specific T cell receptor being expressed by a given cell will be lost. The methods provided by the present invention re-capture TCR sequences from 3′ tag sequencing;
(4) Splice site monitoring from a 3′ tagged cDNA—Similar to above, this method enables any specific sequence within a full-length cDNA to be captured while still maintaining both the entire pool of cDNAs for gene expression analysis, and also maintaining the cell ID sequence from the 3′ tag sequencing library preparation;
(5) Single primer amplification of targeted cDNA (i.e., one round of targeted amplification)—Instead of nested PCR, which is used for targeted RNA sequencing, the present invention allows for performing one round of targeting PCR in order to enrich a cDNA over background such that is not lost and able to be sequenced;
(6) May include modifications to the sequencing assay itself by using gene specific primers in the sequencing reaction to target sequencing from a particular point within a gene-Enables sequencing of key parts of the gene of interest and bypassing certain regions, for example, constant region of the TCR, or being able to sequence multiple splice junctions within a gene;
(7) Compatible with 3′ tag sequencing and DNA conjugated protein analysis at the single cell level;
(8) May use RH-PCR or other modified primer methods for improving PCR specificity—Improves PCR targeting specificity so that only one primer can be used along with a universal primer to amplify the desired product; and/or
(9) Identify transgenes that are not identified by standard single cell RNA-sequencing—Some transgenes are not highly expressed enough to identify in standard RNA sequencing. The methods of the invention can target them specifically for expression analysis.

### Example 1

Enrichment of cDNAs for Sequencing of T Cell Receptor (TCR) Loci from a Single Cell 3′ RNA Sequencing Assay

In this example, a method is shown for enriching cDNAs derived from a 3′ tagged single cell RNA sequencing library, whereby the targeted cDNA is enriched, while maintaining any cell identifying sequences added to the 3′ end of the gene. The method enables, among other things, the sequencing of T cell receptor loci from a single cell 3′ RNA sequencing assay. The assay obtained sequencing data from the TCR as expected.

Rh-primers used for amplifying TCR sequences from TCR alpha and beta chains:

A1
(SEQ ID NO: 1)
ACACTGGCTGCAACAGCATCrCaggaC/3SpC3/
A2
(SEQ ID NO: 2)
GGATAAACATCTGTCTCTGCGrCattgG/3SpC3/
A3
(SEQ ID NO: 3)
AACAGAATGGCCTCTCTGGCrAatcgG/3SpC3/
A4
(SEQ ID NO: 4)
GAACATCACAGCCACCCAGACCGGrAgactG/3SpC3/
A5
(SEQ ID NO: 5)
CTTGGAGAAAGGCTCAGTTCrAagtgA/3SpC3/
A6
(SEQ ID NO: 6)
CTGAGGAAACCCTCTGTGCArGtggaC/3SpC3/
A7
(SEQ ID NO: 7)
AAGGGAATCCTCTGACTGTGrAaatgG/3SpC3/
A8
(SEQ ID NO: 8)
TCCACCAGTTCCTTCAACTTCACCrAtcacT/3SpC3/
A9
(SEQ ID NO: 9)
TTGATACCAAAGCCCGTCTCrAgcacG/3SpC3/
A10
(SEQ ID NO: 10)
TTCATCAAAACCCTTGGGGACAGCrTcatcT/3SpC3/
A11
(SEQ ID NO: 11)
GATGGAAGGTTTACAGCACAGCTCrAatagT/3SpC3/
A12
(SEQ ID NO: 12)
TCCCAGCTCAGCGATTCAGCCTCCrTacatG/3SpC3/
A13
(SEQ ID NO: 13)
CACAGTGGAAGATTAAGAGTCACGCrTtgacT/3SpC3/
A14
(SEQ ID NO: 14)
GCAAAGCTCCCTGTACCTTACGGrCctccG/3SpC3/
A15
(SEQ ID NO: 15)
AATCCGCCAACCTTGTCATCTCCGrCttcaG/3SpC3/
A16
(SEQ ID NO: 16)
ACCCTGAGTGTCCAGGAGGGrTgacaT/3SpC3/
A17
(SEQ ID NO: 17)
CTGAGGAAACCCTCTGTGCArTtggaC/3SpC3/
A18
(SEQ ID NO: 18)
CCCAGCAGGCAGATGATTCTCGTTrAttcgG/3SpC3/
A19
(SEQ ID NO: 19)
CCCAGCAGGCAGATGATTCTCGTTrAttcgG/3SpC3/
A20
(SEQ ID NO: 20)
TCTGGTATGTGCAATACCCCAACCrAaggaG/3SpC3/
A21
(SEQ ID NO: 21)
TTACAAACGAAGTGGCCTCCrCtgttA/3SpC3/
A22
(SEQ ID NO: 22)
GGATTGCGCTGAAGGAAGAGrCtgcaT/3SpC3/
A23
(SEQ ID NO: 23)
AACTGCACGTACCAGACATCrTgggtA/3SpC3/
A24
(SEQ ID NO: 24)
TGAAGGTCACCTTTGATACCACCCrTtaaaG/3SpC3/
A25
(SEQ ID NO: 25)
CACTGCTGACCTTAACAAAGGCGrAgacaA/3SpC3/
A26
(SEQ ID NO: 26)
AAGGAGAGGACTTCACCACGrTactgG/3SpC3/
A27
(SEQ ID NO: 27)
TGCCTCGCTGGATAAATCATCAGGrAcgtaC/3SpC3/
A28
(SEQ ID NO: 28)
AACTGCACGTACCAGACATCrTgggtA/3SpC3/
A29
(SEQ ID NO: 29)
GATAGCCATACGTCCAGATGrTgagtC/3SpC3/
A30
(SEQ ID NO: 30)
TGCCACTCTTAATACCAAGGAGGGrTtacaC/3SpC3/
A31
(SEQ ID NO: 31)
TCTGGTATGTGCAATACCCCAACCrAaggaG/3SpC3/
A32
(SEQ ID NO: 32)
GTCCTGTCCTCTTGATAGCCrTtataA/3SpC3/
A33
(SEQ ID NO: 33)
AGCAAAAACTTCGGAGGCGGrAaataA/3SpC3/
A34
(SEQ ID NO: 34)
ACCCTGCTGAAGGTCCTACATTCCrTgataA/3SpC3/
A35
(SEQ ID NO: 35)
TCCTGGTGACAGTAGTTACGrGgtggT/3SpC3/
A36
(SEQ ID NO: 36)
ACCCTGAGTGTCCAGGAGGGrAgacaC/3SpC3/
A37
(SEQ ID NO: 37)
AGGCTCAAAGCCTTCTCAGCAGGGrAcgatT/3SpC3/
A38
(SEQ ID NO: 38)
GATGGAAGGTTTACAGCACAGCTCrAataaT/3SpC3/
A39
(SEQ ID NO: 39)
AGCCCAGCCATGCAGGCATCTACCrTctgtC/3SpC3/
B1
(SEQ ID NO: 40)
CTCCCTGATTCTGGAGTCCGCCArGcaccT/3SpC3/
B2
(SEQ ID NO: 41)
CCACTCTGAAGATCCAGCCCTCAGrAacccT/3SpC3/
B3
(SEQ ID NO: 42)
CTGTAGCCTTGAGATCCAGGCTACGArAgatC/3SpC3/
B4
(SEQ ID NO: 43)
CTAACATTCTCAACTCTGACTGTGAGCAACArTgagcG/3SpC3/
B5
(SEQ ID NO: 44)
TCGCTCAGGCTGGAGTCGGCTGrCtcccA/3SpC3/
B6
(SEQ ID NO: 45)
TCAAATTTCACTCTGAAGATCCGGTCCACAArAgctgC/3SpC3/
B7
(SEQ ID NO: 46)
GATAACTTCCAATCCAGGAGGCCGAACArCttctA/3SpC3/
B8
(SEQ ID NO: 47)
CCCTGACCCTGGAGTCTGCCArGgcccA/3SpC3/
B9
(SEQ ID NO: 48)
GCTCAGGCTGCTGTCGGCTGrCtcccA/3SpC3/
B10
(SEQ ID NO: 49)
GCTCTGAGCTGAATGTGAACGCCTTGrTtgctT/3SpC3/
B11
(SEQ ID NO: 50)
GCTCTGAGATGAATGTGAGTGCCTTGrGagctC/3SpC3/
B12
(SEQ ID NO: 51)
CCACTCTGACGATTCAGCGCACAGrAgcagG/3SpC3/
B13
(SEQ ID NO: 52)
CCACTCTCAAGATCCAGCCTGCAGrAgatC/3SpC3/
B14
(SEQ ID NO: 53)
CCCCTCAAGCTGGAGTCAGCTGrCtcccA/3SpC3/
B15
(SEQ ID NO: 54)
CTCCCTGTCCCTAGAGTCTGCCATrCcccaT/3SpC3/
B16
(SEQ ID NO: 55)
GCATCCTGAGGATCCAGCAGGTAGrTgcgaC/3SpC3/
B17
(SEQ ID NO: 56)
GCTCTGAGCTGAATGTGAACGCCTTGrTtgctC/3SpC3/
B18
(SEQ ID NO: 57)
CACTCAGGCTGGTGTCGGCTGrCtcccA/3SpC3/
B19
(SEQ ID NO: 58)
GCTCACTTAAATCTTCACATCAATTCCCTGGrAgatC/3SpC3/
B20
(SEQ ID NO: 59)
CCACTCTGAAGATCCAGCCCTCAGrAacccT/3SpC3/
B21
(SEQ ID NO: 60)
CCCTCACGTTGGCGTCTGCTGrTacccA/3SpC3/
B22
(SEQ ID NO: 61)
CCCCTCACTCTGGAGTCTGCTGrCctccA/3SpC3/
B23
(SEQ ID NO: 62)
GCTGGGGTTGGAGTCGGCTGrCtcccA/3SpC3/
B24
(SEQ ID NO: 63)
CCACTCTGAAGATCCAGCGCACAGrAgcagC/3SpC3/
B25
(SEQ ID NO: 64)
CCACTCTCAAGATCCAGCCTGCAGrAgatC/3SpC3/
B26
(SEQ ID NO: 65)
GCTCTGAGCTGAATGTGAACGCCTTGrTtgctC/3SpC3/
B27
(SEQ ID NO: 66)
CCCCCTCACTCTGGAGTCAGCTArCccgcA/3SpC3/
B28
(SEQ ID NO: 67)
CTTATTCCTTCACCTACACACCCTGCrAgccaC/3SpC3/
B29
(SEQ ID NO: 68)
CGCTCAGGCTGGAGTTGGCTGrCtcccA/3SpC3/
B30
(SEQ ID NO: 69)
CATTCTGAACTGAACATGAGCTCCTTGGrAgctgC/3 SpC3/
B31
(SEQ ID NO: 70)
GCTCTGAGATGAATGTGAGCACCTTGrGagctC/3 SpC3/
B32
(SEQ ID NO: 71)
CACTCTGACGATCCAGCGCACACrAgcagC/3 SpC3/
B33
(SEQ ID NO: 72)
CCCTGATCCTGGAGTCGCCCArGccccT/3SpC3/
B34
(SEQ ID NO: 73)
CTTAAACCTTCACCTACACGCCCTGCrAgccaC/3SpC3/
B35
(SEQ ID NO: 74)
CTTGTCCACTCTGACAGTGACCAGTGrCccatG/3 SpC3/
B36
(SEQ ID NO: 75)
CACCTTGGAGATCCAGCGCACAGrAgcagC/3 SpC3/
B37
(SEQ ID NO: 76)
GCTCTGAGCTGAATGTGAACGCCTTGrGagctC/3 SpC3/
B38
(SEQ ID NO: 77)
CAGCCTGGCAATCCTGTCCTCAGrAaccgC/3SpC3/
B39
(SEQ ID NO: 78)
CCACTCTGAAGATCCAGCCCTCAGrAacccT/3SpC3/
B40
(SEQ ID NO: 79)
CTACTCTGAAGGTGCAGCCTGCAGrAactgC/3SpC3/
B41
(SEQ ID NO: 80)
CCTCTCACTGTGACATCGGCCCrAaaagT/3SpC3/
B42
(SEQ ID NO: 81)
CACTCTGACGATCCAGCGCACAGrAgcagG/3SpC3/
B43
(SEQ ID NO: 82)
GCACTCTGAACTAAACCTGAGCTCTCTGrGagctC/3SpC3/
B44
(SEQ ID NO: 83)
CCACTCTGAAGTTCCAGCGCACACrAgcagC/3SpC3/
B45
(SEQ ID NO: 84)
CCTCCTCACTCTGGAGTCCGCTArCcagcA/3SpC3/
B46
(SEQ ID NO: 85)
CTCTACTCTGAAGATCCAGCGCACAGrAgcagC/3SpC3/

The common primer that is currently used for working with 10× genomics libraries is

(SEQ ID NO: 86)
/5InvddT/AATGATACGGCGACCACCGAGATCTACA
CTCTTTCCCTACACGACGCTCTTCCGATCT

RH-PCR TCR A and TCR B

Primer mix comprises:

1. 5′ blocked-TCR (inverted-dT) primer in 100 μM in TE

2. Combine with rhPrimer TCR A (tube labelled A) and TCR B (tube labelled A) mix (25 μM each) 1:1 then dilute 10×

TABLE 1
RHPrimer PCR TCR AB Reaction Mix and PCR conditions
RHPrimer PCR TCR AB Samples: 2

| Component | Volume (μL) | MM (10%) |
|---|---|---|
| 10X PCR Buffer | 2.5 | 5.25 |
| Primer Mix | 0.5 | — |
| (~2.5 μM each) | | |
| 10 mM dNTP | 1 | 2.1 |
| 50 mM MgCl2 | 1.5 | 3.15 |
| Rnase H2 | 0.5 | 1.05 |
| (52 mU/μL) | | |
| cDNA (1:10) | 1 | 2.1 |
| Water | 18 | 37.8 |
| Platinum tar | 0.5 | 1.05 |
| Total | 25 | 52.5 |

3. Aliquot into two samples and add 0.54, of each Primer mix for TCR-A and TCR-B separately.

4. Run Platinum Taq PCR with cycling as shown in Table 2.

TABLE 2
PCR parameters for TCR-A and TCR-B samples.

| PCR | | |
|---|---|---|
| 94° C. | 2 | min |
| 94° C. | 15 | sec |
| 60° C. for TCR-A | 30 | sec |
| 64° C. for TCR-B | | |
| 68° C. | 1 | min |

| | |
|---|---|
| 4° C. | forever |

5. Cycle for 25 cycles.

6. Ampure cleanup with 1:1 ratio and wash with 80% ETHO, elute with 20 μL of EB buffer (10 mM Tris pH 8.5).

PCR result is shown in FIG. 7.

7. Because platinum Taq adds “A” to the ends, all that is needed to do is to add PNK to phosphorylate the 5′ end.

8. Prepare reaction as follows in Table 3 for each sample

TABLE 3
PNK Reaction mixture.
PNK ReacYon

| Component | Volume (μL) |
|---|---|
| | 40 |
| 10X T4 DNA ligase buffer | 4 |
| Library | 20 |
| PNK | 2 |
| H2O | 14 |

37 degrees Celsius for 30 minutes

65 degrees Celsius for 20 minutes

Ligate Adapters:

TABLE 4
Ligation Reaction.
Ligation Reaction

| Component | Volume (μL) |
|---|---|
| 2X Quick Ligase Buffer | 50 |
| PNK Reaction | 40 |
| Quick Ligase | 3 |
| JS Annealed Adapter | 1 |
| (50 μM) | |
| H2O | 6 |

9. Ligate at room temperature for 15 minutes.

10. Ampure cleanup with 1:1 ratio and wash with 80% ETOH, elute with 20 μL of EB buffer (10 mM Tris pH 8.5)

11. Use 10 μL of elution for PCR.

Library Enrichment:

TABLE 5
Library Enrichment Reaction and PCR parameters.
Library Enrichment

| Component | Volume (50 μL) | 3.1X |
|---|---|---|
| 2X MyTaq Reaction | 25 | 77.5 |
| Ligated Reaction | 10 | 10 |
| JS296 | 1.5 | 4.65 |
| JS289 | 1.5 | 4.65 |
| H2O | 12 | 37.2 |

| PCR | | |
|---|---|---|
| 95° C. | 1 | min |
| 95° C. | 15 | sec |
| 60° C. | 1 | sec |
| 72° C. | 10 | s |

| | |
|---|---|
| 4° C. | forever |

12 cycles

### Results

The TCR alpha and TCR beta RNAs were PCR amplified using the described assays and results are shown in FIG. 9. In lanes 4 and 5, the PCR products show the correct size (with an additional band in lane 5, lower MW). Subsequent to the PCR, the Illumina® adaptor was ligated as described and the correct bands can be seen in both TCR alpha and beta (lanes 2 and 3).

Similar methods have been used to amplify specific CAR transgenes, but instead of ligating the Illumina® adaptor, an indexing PCR step is used. Identification of the CAR sequence requires the PCR methods because the transgene on its own is not sequenced to sufficient depth to identify which cells are CAR positive. While it is possible that significantly deeper sequencing would solve this problem, the described method is far less expensive and is more robust.

### Example 2

Enrichment of cDNAs for Sequencing of T Cell Receptor Alpha and Beta Using Pooled Primer PCR

Using an embodiment of the general method depicted in FIG. 3, T cell receptor alpha (TCRa) and beta (TCRb) cDNAs were enriched by individual and pooled primer PCR amplification to enable subsequent sequencing. TCR primers were initially tested individually and then pooled. The following materials and methods were employed in these experiments.

### Methods

### Individual Primer PCR

cDNA input was cDNA derived from human PBMCs run through 10× Genomics 3′ RNAseq assay following recommended directions.

PCR conditions: 25 μl reactions using 1U of platinum taq (Thermofisher Scientific, Waltham, Mass.), 1×PCR buffer (Thermofisher Scientific), 1.5 mM MgCl₂, 0.52mU RNAseH2 (IDT), 100 nM each primer (Integrated DNA Technologies, Inc. (IDT), Coralville, Iowa). PCR reaction: 94° C., 2 min, then 30 cycles of 94° C. 15 sec, 60° C. (TCR alpha)/64° C. (TCR beta) 30 sec, 68° C. 1 min.

Sequencing was performed on MiSeq (Illumina, Inc., San Diego, Calif.) using 100 base read 1 and 400 base read 2. Data were analyzed using MIXCR: Bolotin, D., Poslaysky, S., Mitrophanov, I. et al. MiXCR: software for comprehensive adaptive immunity profiling. Nat Methods 12, 380-381 (2015) doi:10.1038/nmeth.3364.

### Pooled Primer PCR

Individual TCR alpha primers were pooled at 25 nM each. Separately, TCR beta primers were pooled at 25 nM each. A single PCR for each of TCR alpha and TCR beta was performed.

PCR conditions: 25 μl reactions using 1U of platinum taq (Thermofisher Scientific), 1×PCR buffer (Thermofisher Scientific), 1.5 mM MgCl₂, 0.52mU RNAseH2 (Integrated DNA Technologies, Inc. (IDT), Coralville, Iowa), 100 nM reverse common primer (IDT) and a pool of 25 nM each TCR primer. PCR reaction: 94° C., 2 min, then 30 cycles of 94° C. 15 sec, 60 C (TCR alpha)/64° C. (TCR beta) 30 sec, 68° C. 1 min.

### Purification and Sequencing of PCR Products

PCR products were purified using Ampure beads (Beckman Coulter Life Sciences, Indianapolis, Ind.) at a ratio of 1:1. PCR products were treated with polynucleotide kinase and ligated to sequencing adapters using the Quick Ligation Kit (NEB, Ipswich, Mass.) at 37 C for 30 min followed by 65C for 20 minutes. PCR product was ligated to the following double stranded oligo: 5′ GATCGGAAGAGCACACGT (SEQ ID NO: 87) and 5′ GTGACTGGAGTTCAGACGTGTGCTCTTCCGATCT (SEQ ID NO: 88).

Ligation product was purified with Ampure at a 1:1 ratio, and subsequently PCR amplified using MyTaq (Bioline Meridian Bioscience, Memphis, Tenn.) in a 25 μl reaction with 200 nM each of AATGATACGGCGACCACCGAGATCTACACTCTTTCCCTACACGACGCTCTTCCGA T*C*T (SEQ ID NO: 89), and CAAGCAGAAGACGGCATACGAGATCGTCCATTGTGACTGGAGTTCAGACGTGTG CTCTTC (SEQ ID NO: 90), where *=phosphorothioate bond. PCR conditions were: 95° C. 1 min, followed by 12 cycles of 95° C., 15 sec, 60° C., 1 min, 72° C., 10 sec.

PCR products were purified with Ampure at a 1:1 ratio and used for sequencing on MiSeq (Illumina) with the run parameters: read 1, 100 bases, read 2, 400 bases. Reads were trimmed for quality and resulting data was analyzed using MIXCR: Bolotin, D., Poslaysky, S., Mitrophanov, I. et al. MiXCR: software for comprehensive adaptive immunity profiling. Nat Methods 12, 380-381 (2015) doi:10.1038/nmeth.3364.

The MIXCR output data is summarized below.

MIXCR output data:

Analysis Date: Tue Apr 23 04:16:01 UTC 2019

Input file(s):

/data/input/samples/231750570/JS296_S1_L001_R1_001.fastq.gz/data/input/samples/231750 570/JS296_S1_L001_R2_001.fastq.gz

Output file:

Command line arguments:

Total sequencing reads: 4867049

Successfully aligned reads: 80353

Successfully aligned, percent: 1.65%

Alignment failed because of absence of V hits: 4.53%

Alignment failed because of absence of J hits: 89.94%

Alignment failed because of low total score: 3.88%

Overlapped, percent: 7.72%

Overlapped and aligned, percent: 0.33%

Overlapped and not aligned, percent: 7.39%

Analysis Date: Tue Apr 23 04:16:04 UTC 2019

Input file(s):

Output file:

Command line arguments:

Final clonotype count: 2025

Total reads used in clonotypes: 5678

Reads used, percent of total: 0.12%

Reads used as core, percent of used: 50.16%

Mapped low quality reads, percent of used: 49.84%

Reads clustered in PCR error correction, percent of used: 0.12%

Clonotypes eliminated by PCR error correction: 7

Percent of reads dropped due to the lack of clonal sequence:

Percent of reads dropped due to low quality: 0%

Percent of reads dropped due to failed mapping: 0.17%

### Results

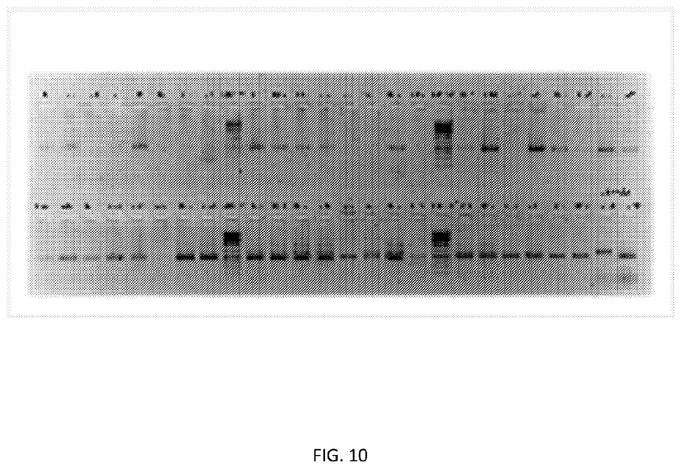

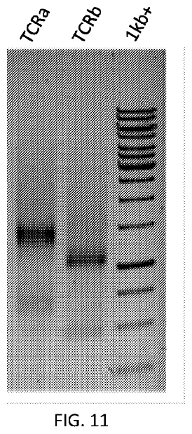

Both individual (FIG. 10) and pooled (FIG. 11) primer PCR reactions were used successfully in an example of the methods described herein to enrich for TCR alpha and TCR beta sequences in cDNA derived from human PBMCs. The resulting amplicons from the PCR reactions were subsequently purified and analyzed to confirm the presence of TCR alpha and TCR beta sequences.

As used herein, the indefinite articles “a” and “an” should be understood to mean “at least one” unless clearly indicated to the contrary.

The phrase “and/or”, as used herein, should be understood to mean “either or both” of the elements so conjoined, i.e., elements that are conjunctively present in some cases and disjunctively present in other cases.

It should also be understood that, unless clearly indicated to the contrary, in any methods described herein that include more than one step or act, the order of the steps or acts of the method is not necessarily limited to the order in which the steps or acts of the method are recited.

Unless otherwise indicated or otherwise evident from the context and understanding of one of ordinary skill in the art, values that are expressed as ranges can assume any specific value or subrange within the stated ranges in various embodiments, unless the context clearly dictates otherwise.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of enriching a target complementary DNA (cDNA), comprising the steps of:
(a) providing a plurality of different cDNAs, wherein each cDNA comprises a first universal sequence at an end, and wherein the plurality of cDNAs comprises the target cDNA to be enriched;
(b) providing a first reaction mixture comprising:
1) a first universal oligonucleotide forward primer comprising a sequence that is complementary to all or a portion of the first universal sequence, and
2) at least one gene specific reverse primer, wherein the at least one gene specific reverse primer comprises a sequence that is complementary to all or a portion of a sequence in the target cDNA, and further comprises at least one second universal sequence at an end of the at least one gene specific primer;
(c) contacting the plurality of cDNAs with the first reaction mixture;
(d) amplifying the target cDNA to obtain an amplicon;
(e) providing a second reaction mixture comprising:
1) a second universal oligonucleotide forward primer comprising a sequence that is complementary to all or a portion of the first universal sequence, and
2) a universal oligonucleotide reverse primer, wherein the universal oligonucleotide reverse primer comprises a sequence that is complementary to all or a portion of the at least one second universal sequence; thereby enriching for the target cDNA;
(f) contacting the amplicon with the second reaction mixture; and
(g) amplifying the amplicon, thereby enriching for the target cDNA.

2. The method of claim 1, wherein each cDNA in the plurality of cDNAs further comprises a cell identification tag or a unique molecular identifier (UMI) sequence, or a combination thereof.

3. The method of claim 2, wherein the cell identification tag or UMI sequence, or combination thereof, further comprises a poly(T) sequence.

4. The method of any one of claims 1-3, wherein the first universal sequence is at the 5′ end of each cDNA molecule.

5. The method of any one of claims 1-3, wherein the first universal sequence is at the 3′ end of each cDNA molecule.

6. The method of any one of claims 1-5, wherein the plurality of cDNAs is obtained by reverse transcribing mRNA from a single cell.

7. The method of any one of claims 1-6, wherein amplifying the target cDNA in (d) comprises amplifying by polymerase chain reaction.

8. The method of any one of claims 1-6, wherein the at least one gene specific reverse primer further comprises a blocking domain separated from the primer by an RNA base.

9. The method of claim 8, wherein amplifying the target cDNA in (d) comprises amplifying by an RNase H-dependent polymerase chain reaction.

10. The method of any one of claims 2-9, wherein the cell identification tag or the UMI sequence, or the combination thereof, are preserved after the target cDNA is amplified.

11. The method of any one of claims 1-10, further comprising sequencing one or more portions of the target cDNA using the universal oligonucleotide forward primer, the universal oligonucleotide reverse primer, the at least one gene specific reverse primer, or a combination thereof.

12. A method of enriching a target complementary DNA (cDNA), comprising the steps of:
(a) providing a plurality of cDNAs, wherein each cDNA comprises a first universal sequence at an end, and wherein the plurality of cDNAs comprises the target cDNA to be enriched;
(b) providing a first reaction mixture comprising:
1) a universal oligonucleotide forward primer comprising a sequence that is complementary to all or a portion of the first universal sequence, and
2) a gene specific reverse primer comprising a sequence that is complementary to a sequence in the target cDNA;
(c) contacting the plurality of cDNAs with the first reaction mixture;
(d) amplifying the target cDNA to obtain a first amplicon;
(e) adding at least one second universal sequence to an end of each target cDNA in the first amplicon to obtain a conjugated amplicon;
(f) providing a second reaction mixture comprising:
1) a universal oligonucleotide forward primer comprising a sequence that is complementary to all or a portion of the first universal sequence,
2) a universal oligonucleotide reverse primer comprising a sequence that is complementary to all or a portion of the at least one second universal sequence;
(g) contacting the conjugated amplicon with the second reaction mixture; and
(h) amplifying the conjugated amplicon, thereby enriching for the target cDNA.

13. The method of claim 12, wherein each cDNA in the plurality of cDNAs further comprises a cell identification tag or a unique molecular identifier (UMI) sequence, or a combination thereof.

14. The method of claim 13, wherein the cell identification tag or UMI sequence, or combination thereof, further comprises a poly(T) sequence.

15. The method of any one of claims 12-14, wherein the first universal sequence is at the 5′ end of each cDNA molecule.

16. The method of any one of claims 12-14, wherein the first universal sequence is at the 3′ end of each cDNA molecule.

17. The method of any one of claims 12-16, wherein the plurality of cDNAs is obtained by reverse transcribing mRNA from a single cell.

18. The method of any one of claims 12-17, wherein at least one of the amplifying steps comprises amplifying by polymerase chain reaction.

19. The method of any one of claims 12-17, wherein the gene specific reverse primer further comprises a blocking domain separated from the primer by an RNA base.

20. The method of claim 19, wherein at least one of the amplifying steps comprises amplifying by an RNase H-dependent polymerase chain reaction.

21. The method of claim 12, wherein the at least one second universal sequence is ligated to an end of the first amplicon opposite the first universal sequence.

22. The method of claim 12, wherein one end of the universal oligonucleotide forward primer has a blocking group, thereby blocking phosphorylation of the universal oligonucleotide forward primer on the one end.

23. The method of claim 22, wherein the blocking group is an inverted dTTP.

24. The method of any one of claims 22-23, wherein the blocking group is at the 5′ end of the universal oligonucleotide forward primer.

25. The method of claim 12, wherein the at least one second universal sequence is added using transposons.

26. The method of claim 12, wherein the at least one second universal sequence is added by ligating the at least one second universal sequence to an end of each target cDNA in the first amplicon, by a primer extension reaction, or by a nucleic acid amplification reaction, or a combination thereof.

27. The method of claim 12, wherein the at least one second universal sequence is added by fragmenting nucleic acid molecules in the first amplicon and ligating the at least one second universal sequence to the fragments, by a primer extension reaction, or by a nucleic acid amplification reaction, or a combination thereof.

28. The method of claim 12, wherein the at least one second universal sequence is added at an end opposite to the at least one first universal sequence.

29. The method of any one of claims 12-28, wherein the cell identification tag or the UMI sequence, or the combination thereof, are preserved after the conjugated amplicon of target cDNA is amplified.

30. The method of any one of claims 12-29, further comprising sequencing one or more portion of the target cDNA using the universal oligonucleotide forward primer, the universal oligonucleotide reverse primer, the gene specific reverse primer, a gene specific forward primer, or a combination thereof.

31. A method of enriching a target complementary DNA (cDNA), comprising the steps of:
(a) providing a plurality of cDNAs, wherein each cDNA comprises a first universal sequence at an end and wherein the plurality of cDNAs comprises the target cDNA to be enriched;
(b) providing a first reaction mixture comprising:
at least one gene specific primer comprising a sequence that is complementary to all or a portion of a sequence in the target cDNA, and further comprises at least one second universal sequence at an end of the at least one gene specific primer;
(c) contacting the plurality of cDNAs with the first reaction mixture such that the at least one gene specific primer hybridizes with the target cDNA;
(d) extending the at least one gene specific primer to obtain at least one extension product;
(e) providing a second reaction mixture comprising:
1) a universal oligonucleotide forward primer comprising a sequence that is complementary to all or a portion of the first universal sequence; and
2) a universal oligonucleotide reverse primer comprising a sequence that is complementary to all or a portion of the at least one second universal sequence;
(f) contacting the at least one extension product with the second reaction mixture; and
(g) amplifying the at least one extension product, thereby enriching for the target cDNA.

32. The method of claim 31, wherein each cDNA in the plurality of cDNAs further comprises a cell identification tag or a unique molecular identifier (UMI) sequence, or a combination thereof.

33. The method of claim 32, wherein the cell identification tag or UMI sequence, or combination thereof, further comprises a poly(T) sequence.

34. The method of any one of claims 31-33, wherein the first universal sequence is at the 5′ end of each cDNA molecule.

35. The method of any one of claims 31-33, wherein the first universal sequence is at the 3′ end of each cDNA molecule.

36. The method of any one of claims 31-35, wherein the plurality of cDNAs is obtained by reverse transcribing mRNA from a single cell.

37. The method of any one of claims 31-36, wherein amplifying the at least one extension product in (g) comprises amplifying by polymerase chain reaction.

38. The method of any one of claims 31-37, wherein the cell identification tag or the UMI sequence, or the combination thereof, are preserved after the at least one extension product is amplified.

39. The method of any one of claims 31-38, further comprising sequencing one or more portion of the target cDNA using the universal oligonucleotide forward primer, the universal oligonucleotide reverse primer, the gene specific primer, or a combination thereof.

40. The method of any one of claims 31-39, wherein the first reaction mixture comprises a polymerase deficient in flap endonuclease activity.

41. The method of any one of the preceding claims, further comprising the step of sequencing the enriched target cDNA.

42. A method of enriching a target complementary DNA (cDNA), comprising the steps of:
(a) providing a plurality of different cDNAs, wherein each cDNA comprises a first universal sequence at an end, and wherein the plurality of cDNAs comprises the target cDNA to be enriched;
(b) providing a reaction mixture comprising:
1) a universal oligonucleotide forward primer comprising a sequence that is complementary to all or a portion of the first universal sequence,
2) at least one gene specific reverse primer, wherein the at least one gene specific reverse primer comprises a sequence that is complementary to all or a portion of a sequence in the target cDNA, and further comprises at least one second universal sequence at an end of the at least one gene specific primer, and
3) a universal oligonucleotide reverse primer, wherein the universal oligonucleotide reverse primer comprises a sequence that is complementary to all or a portion of the at least one second universal sequence;
(c) contacting the plurality of cDNAs with the reaction mixture; and
(d) amplifying the target cDNA, thereby enriching for the target cDNA.

43. The method of claim 42, wherein each cDNA in the plurality of cDNAs further comprises a cell identification tag or a unique molecular identifier (UMI) sequence, or a combination thereof.

44. The method of claim 43, wherein the cell identification tag or UMI sequence, or combination thereof, further comprises a poly(T) sequence.

45. The method of any one of claims 42-44, wherein the first universal sequence is at the 5′ end of each cDNA molecule.

46. The method of any one of claims 42-44, wherein the first universal sequence is at the 3′ end of each cDNA molecule.

47. The method of any one of claims 42-46, wherein the plurality of cDNAs is obtained by reverse transcribing mRNA from a single cell.

48. The method of any one of claims 42-47, wherein amplifying the target cDNA in (d) comprises amplifying by polymerase chain reaction.

49. The method of any one of claims 42-47, wherein the at least one gene specific reverse primer further comprises a blocking domain separated from the primer by an RNA base.

50. The method of claim 49, wherein amplifying the target cDNA in (d) comprises amplifying by an RNase H-dependent polymerase chain reaction.

51. The method of any one of claims 43-50, wherein the cell identification tag or the UMI sequence, or the combination thereof, are preserved after the target cDNA is amplified.

52. The method of any one of claims 42-51, further comprising sequencing one or more portions of the target cDNA using the universal oligonucleotide forward primer, the universal oligonucleotide reverse primer, the at least one gene specific reverse primer, or a combination thereof.
